(19) United States
(12) Patent Application Publication
(10) Pub. No.: US 2007/0099881 A1
(43) Pub. Date: May 3, 2007

(54) Method of treating wounds by enhancing expression of procollagen

(76) Inventors: Norman Barton, Phoenix, ND (US); Theodore Kramer, Short Hills, NJ (US)

Correspondence Address: KENYON & KENYON LLP, ONE BROADWAY, NEW YORK, NY, 10004, US

(21) Appl. No.: 11/606,446

(22) Filed: Nov. 29, 2006

Related U.S. Application Data
(62) Division of application No. 10/058,066, filed on Jan. 29, 2002.
(60) Provisional application No. 60/264,255, filed on Jan. 29, 2001.

(51) Int. Cl. A61K 31/57

(52) U.S. Cl. 514/169

(57) ABSTRACT

The present invention provides methods for identifying compounds useful for the treatment of wounds by determining whether the compound increases the expression of type I procollagen and/or type III procollagen by human fibroblasts cultured in the presence of collagen. The present invention also provides methods for treating wounds by applying to the wound and/or surrounding tissue a therapeutically effective amount of anabolic steroid in a pharmaceutically acceptable formulation, wherein the steroid is not administered systemically to a patient. The present invention also provides compositions comprising at least one anabolic steroid in an amount effective to promote collagen production in at the site of a wound, wherein the composition is a pharmaceutically acceptable formulation suitable for local administration.

### PRIORITY

This application claims priority from provisional application 60/264,255 filed Jan. 29, 2001.

### BACKGROUND OF THE INVENTION

This invention relates to the treatment of wounds using anabolic steroids, particularly oxandrolone, which increase expression of procollagen in a wound or surrounding tissue.

Oxandrolone

Oxandrolone (17-methyl-17-hydroxy-2-oxa-5-androstan-3-one) is an anabolic steroid synthetically derived from testosterone. The preparation of oxandrolone is described, at least in U.S. Pat. No. 3,128,283, and oxandrolone is commercially available. (Oxandrin® from Bio-Technology General Corp., Iselin, N.J.). Oxandrolone has a unique chemical structure compared with other testosterone analogs in that it contains an oxygen rather than a carbon atom at the 2-position within the phenanthrene nucleus (Fox and Minor, “Oxandrolone, a potent anabolic steroid,” J. Clin. Endocrinol. Metab. 22: 921-924(1962)) and lacks a 4-ene function in the A-ring.

Effects of Oxandrolone on Weight Gain and Reversal of Catabolic State

In vivo studies report that anabolic steroids promote weight gain. Clinically, anabolic steroids restore lean body mass and muscle lost as a consequence of severe trauma, including burns, or chronic infections, such as HIV. See, e.g., Demling and DeSanti, “Oxandrolone, an anabolic steroid, significantly increases the rate of weight gain in the recovery phase after major burns,” J. Trauma 43: 47-50 (1997); Strawford et al., “Resistance exercise and supraphysiologic androgen therapy in eugonadal men with HIV-related weight loss: A randomized controlled trial,” JAMA 281: 1282-1290 (1999); Hansmann et al., “Anabolic steroids in polytrauma patients: influence on renal nitrogen and amino acid losses; a double blind study,” J. Parent. Enteral. Nutr. 14: 111-114 (1990).

Although it has been noted that weight gain in patients who are suffering from involuntary weight loss or who are in a catabolic state treated with anabolic steroids may be accompanied by wound closure, the wound closing effect has been attributed to a reversal of a generalized catabolic state, which reversal allowed the body to devote more resources to protein synthesis and wound healing. See, e.g., Uttupa and Chansoria, “Studies on wound healing,” Indian J. Med. Res. 57: 447-55 (1969); Nair et al. (1977); Ehrlich and Hunt (1969). See, also, Da Lio A L et al., “Oxandrolone ameliorates the adverse effects of corticosteroids in wound healing,” Surgical Forum pp. 628-620 (1999); Demling, “Oxandrolone, an anabolic steroid, enhances the healing of a cutaneous wound in the rat,” Wound Rep. Regen. 8: 97-102 (2000); Demling, “Comparison of the anabolic effects and complications of human growth hormone and the testosterone analog, oxandrolone after severe burn injury,” Burns 25: 215-20 (1999).

Inhibition of Collagen Production by Cells Grown on Collagen

The wound repair process entails the re-establishment of a connective tissue matrix, a scar, in which collagen is the major matrix component. Collagen deposition is critical for initial gains in wound breaking strength and makes up the extracellular matrix of granulation tissue. Cells first produce procollagen, which is then converted to collagen. Thus, procollagen production is important for wound healing, and methods and compounds capable of increasing procollagen production by fibroblasts in and around healing wounds would find use in clinical settings wherein wound healing is desirable. Additionally, assays useful for identifying agents and methods for increasing procollagen production by fibroblasts would be useful in identifying clinically useful methods and compositions.

The effects that anabolic steroids have on expression of procollagen and collagen synthesis by fibroblasts grown on collagen or in a collagen-enriched environment have not previously been investigated.

### SUMMARY OF THE INVENTION

The subject invention provides methods and compounds for the treatment of wounds by administering formulations comprising a therapeutically effective amount of an anabolic steroid. More particularly, the subject invention provides methods and compounds for the treatment of wounds comprising administering formulations comprising an anabolic steroid, wherein that anabolic steroid is selected and administered in an amount effective to increase expression of procollagen, particularly procollagen type I and/or procollagen type III, in a wound and/or surrounding tissue to therapeutic levels.

Preferred anabolic steroids for use in the methods and compositions of the present invention include those that have high anabolic to androgenic ratios. In particular for systemic administration, preferred steroids include those that have low toxicity, particularly low hepatotoxicity. However, where the mode of administration is topical, steroids which have greater toxicity when administered systemically may also be used, and may be desirable where they are more effective at wound healing than are anabolic steroids with low toxicity.

A preferred anabolic steroid for use in the methods and compositions of the present invention is oxandrolone.

The present invention also provides methods for identifying compounds useful for the treatment of wounds by determining whether a compound of interest increases the expression of type I procollagen and/or type III procollagen by fibroblasts cultured in the presence of collagen. In certain embodiments, compounds which increase the expression of both type I and type III collagen by fibroblasts grown on collagen are selected. In certain embodiments, compounds which increase the expression of type I procollagen and/or type III procollagen by human fibroblasts grown on collagen are selected.

The subject invention provides methods and compounds for the treatment of wounds by administering formulations comprising a therapeutically effective amount of a compound which was identified by an assay or assays to determine whether a compound of interest increases the expression of type I procollagen and/or type III procollagen by human fibroblasts cultured in the presence of collagen. The present invention provides methods and compounds for the treatment of wounds. Such compounds are selected by the inventive methods for selective compounds useful for treatment of wounds, and the methods comprise administration of therapeutically effective amounts of such compounds. The identified compound is preferably administered in an amount effective to increase expression of procollagen by a wound and/or surrounding tissue to therapeutic levels.

In certain embodiments of the subject invention, more than one compound according to the present invention is administered to a patient. Where more than one compound is administered, the compounds may be administered sequentially or simultaneously.

In certain embodiments of the present invention, a compound according to the present invention is administered systemically. In certain of these embodiments, a compound according to the present invention is administered orally. In other embodiments, a compound according to the present invention is administered by intravenous injection or intramuscular injection. In other embodiments, a compound according to the present invention is administered rectally or intravaginally.

The present invention provides methods for treating wounds comprising administration of a therapeutic amount of a compound according to the present invention in pharmaceutically acceptable formulations suitable for local administration. In certain embodiments of the present invention, pharmaceutically acceptable formulations suitable for injection into tissues comprising the wound and/or tissues bordering a wound are provided. In certain embodiments of the present invention, pharmaceutically acceptable formulations suitable for topical application onto said wound and/or surrounding tissues are provided.

The present invention also provides methods and compositions for treating a wound or wounds comprising the step of applying to said wound a therapeutically effective amount of a compound according to the subject invention in a pharmaceutically acceptable formulation, wherein said compound is not administered systemically to a patient.

The present invention also provides methods and compositions for treating a wound or wounds comprising the step of applying to said wound a therapeutically effective amount of a compound according to the subject invention in a pharmaceutically acceptable formulation, wherein said compound is administered locally in an amount effective to accelerate wound healing, wherein this effective amount is less than the amount of the compound effective to accelerate wound healing when administered systemically.

Wounds that may be treated using the compounds and methods of the present invention include pressure ulcers, incisional wounds, traumatic wounds, diabetic ulcers, ischemic ulcers, venous ulcers, burns, and internal wounds and injuries, such as internal surgical incisions and anastomoses, gastric ulcers and internal bruising. In certain embodiments, wounds that are treated according to the present invention are wounds produced by external trauma or forces, such as, but not limited to, incisional wounds, surgical wounds, traumatic wounds and wounds caused by accidents, pressure ulcers, and the like.

In certain embodiments, the wounds are not atherosclerotic lesions, ocular lesions, or immunopathological lesions in lacrimal tissue. In certain embodiments, wounds treated according to the present invention are not burns. In certain embodiments, the wounds treated according to the present invention are not surgical incisions.

In certain embodiments, wounds that were caused by a force or occurrence external to a patient's body are treated by administration of a compound according to the present invention. By “a force or occurrence external to a patient's body”, it is meant an accidental or intentional act or condition that wounds the patient's body, such as the making of a surgical incision, trauma caused by, for example, a fall or an automobile or other accident, or a deliberate assault, or a sore or abrasion caused by excess or prolonged pressure, such as a bed sore. The phrase “a force or occurrence external to a patient's body”, is not meant to encompass disorders or diseases caused by, for example, infectious agents or malfunctions of the patient's body due to, for example, genetic disorders.

In certain embodiments of the present invention, methods and compounds of the present invention are used to treat a wound or wounds that are present in a patient whose body weight is less than their ideal body weight. In certain embodiments of the present invention, methods and compounds of the present invention are used to treat a wound or wounds that are present in a patient who is experiencing or who has recently experienced involuntary weight loss or chronic wasting or who is or recently was in a catabolic state.

In certain other embodiments of the present invention, methods and compounds of the present invention are used to treat a wound or wounds that are present in a patient whose body weight is equal to or greater than their ideal body weight. In certain embodiments of the present invention, methods and compounds of the present invention are used to treat a wound or wounds that are present in a patient who is not and has not recently been in a catabolic state. In certain embodiments of the present invention, methods and compounds of the present invention are used to treat a wound or wounds that are present in a patient who is not experiencing and has not recently experienced involuntary weight loss or chronic wasting.

In certain embodiments of the present invention, methods and compounds of the present invention are used to treat a wound or wounds that are present in a patient who is not suffering from and has not recently suffered from an autoimmune disorder or disease, such as HIV infection or AIDS, multiple sclerosis, or keratoconjunctivitis sicca (KCS), such as Sjogren's syndrome. In certain embodiments of the present invention, methods and compounds of the present invention are used to treat a wound or wounds that are present in a patient who is not suffering from and has not recently suffered from chronic obstructive pulmonary disease, an infectious disease, particularly a chronic infection, which has caused or is causing involuntary weight loss, extensive surgery or severe trauma that has caused or is causing involuntary weight loss, alcoholic hepatitis, Turner's syndrome, constitutional delay of growth and puberty in boys, or Facioscapulohumeral Dystrophy (FSHD). However, in certain embodiments, methods and compounds of the present invention are administered topically to treat a wound or wounds that are present in a patient suffering from a condition as described in this paragraph.

In certain embodiments, compounds of the present invention are administered to patients who have wounds that are new, and not yet experiencing delayed healing. In certain embodiments, compounds of the present invention are administered to patients who have wounds that are not associated with neovascularization. In certain embodiments of the invention, compounds of the present invention are administered in doses that neither prevent nor treat neovascularization. In certain embodiments, compounds of the present invention are administered to patients who have wounds that are not associated with head trauma, spinal trauma, septic or traumatic shock, stroke, hemorrhagic shock, cancer, arthritis, arteriosclerosis, angiofibroma, arteriovenous malformations, corneal graft neovascularization, delayed wound healing, diabetic retinopathy, granulations, burns, hemangioma, hemophilic joints, hypertrophic scars, neovascular glaucoma, nonunion fractures, Osler-Weber Syndrome, psoriasis, pyogenic granuloma, retrolental fibroplasia, scleroderma, solid tumors, trachoma, vascular adhesions, pterigium, or solid tumor growth.

In certain embodiments of the present invention, a compound of the present invention is systemically administered to patients in a dosage which is less than the dosage which would be administered to promote gain of weight, lean mass, and/or muscle mass in said patient.

In certain embodiments, a wound or wounds are treated in a patient who is or has recently been treated with corticosteroids. The subject invention provides methods and compounds for reversing, in part or in whole, the catabolic effects of corticosteroids.

In other embodiments of the present invention, a wound or wounds are treated in a patient who is not being and who has not recently been treated with corticosteroids.

### BRIEF DESCRIPTION OF THE FIGURES

FIG. 1. Northern blots of type I procollagen and GAPDH mRNAs. RNA was extracted from HF 250 cells grown on collagen or plastic surfaces in the presence or absence of anabolic steroid for 24 hours. Type I procollagen mRNA expression was less in fibroblasts grown on collagen (lane A) in comparison to fibroblasts grown on plastic (lane C). The mRNA from HF 250 cells plated on collagen in the presence of oxandrolone is shown in lane B. Lane D shows mRNA from cells growing on plastic receiving oxandrolone. The middle panel shows the GAPDH mRNA expression as a measure of RNA loading and transfer. The bottom panel is a bar graph presentation of the ratio of procollagen mRNA density with that of GAPDH mRNA density.

FIG. 2. Northern blots of type III procollagen and GAPDH mRNAs. RNA was extracted from HF 250 cells grown on collagen or plastic surfaces in the presence or absence of oxandrolone for 24 hours. Type III collagen mRNA expression in untreated fibroblasts grown on collagen is shown in lane A and untreated cells grown on plastic shown in lane C. Oxandrolone enhanced the expression of type III procollagen mRNA in cells grown on collagen (lane B) and in cells grown on plastic (lane D). The middle panel shows the GAPDH mRNA expression as a measure of RNA loading and transfer. The bottom panel is a bar graph presentation of the ratio of procollagen mRNA density with that of GAPDH mRNA density. DC01 355356 v 1

FIG. 3. Ideal Body Weight for Height Table: Ideal body weight for height can be determined by locating on the table a patient's height in the appropriate column for the patient's sex, then reading the corresponding ideal body weight.

### DETAILED DESCRIPTION OF THE INVENTION

The subject invention provides methods and compounds for the treatment of wounds by administering formulations comprising a therapeutically effective amount of anabolic steroid. More particularly, the subject invention provides methods and compounds for the treatment of wounds comprising administering formulations comprising an anabolic steroid, wherein that anabolic steroid is selected and administered in an amount effective to increase expression of procollagen, particularly procollagen type I and/or procollagen type III, in a wound and/or surrounding tissue to therapeutic levels.

The subject invention provides methods for selecting compounds useful for wound healing, comprising evaluating the capability of the candidate compound to increase the expression of procollagen, particularly procollagen type I and/or procollagen type III, fibroblasts growing in a collagen-rich environment. The present invention provides methods and compounds for the treatment of wounds. Such compounds are selected by the inventive methods for selective compounds useful for treatment of wounds, and the methods comprise administration of therapeutically effective amounts of such compounds.

As administration of anabolic steroids such as oxandrolone in amounts according to the present invention is sufficient to increase expression of procollagen in a wound and/or surrounding tissues independent of systemic reversal of a catabolic state or reversal of involuntary weight loss, anabolic steroids may be used to heal wounds in patients whose weight is equal to or greater than their ideal weight and who are not suffering from a catabolic state. Such treatment of wounds in such patients has not previously been undertaken.

As a systemic response is not required for anabolic steroids such as oxandrolone to increase wound healing, anabolic steroids may be applied topically to a wound and/or the surrounding area. Such topical application would eliminate systemic side effects associated with systemic treatment with anabolic steroids. Accordingly, anabolic steroids may be used at earlier stages than they were previously used. Further, anabolic steroids may be administered topically to wound patients to whom they would not be administered systemically due to particular sensitivity to anabolic steroids or contraindications to systemic use of anabolic steroids. Examples of such patients include those suffering from disorders of the kidney. Additionally, a wider range of anabolic steroids may be used to treat wound healing, including those steroids that have exhibit greater toxicity when administered systemically than those anabolic steroids typically used for systemic treatment. Such use of more toxic steroids may be desired, inter alia, where such steroids have greater wound healing properties than less toxic steroids.

The present invention also provides a new method for evaluating the effectiveness of a compound in promoting wound healing. The results of the Examples provided herein demonstrate the evaluation of compounds for their ability to promote synthesis of type I and type III procollagen (the primary two types of collagen found in skin) by fibroblasts grown on collagen. Unlike fibroblasts growing on plastic, fibroblasts growing on collagen are growing in conditions similar to those found in a healing wound in vivo. Thus, the inventive technique is valuable for identifying compounds useful for treating wounds in vivo.

Human dermal fibroblasts were grown on collagen-coated or uncoated plastic dishes in the presence of ascorbic acid to optimize collagen processing. By Northern Blot analysis, fibroblasts growing on collagen expressed less types I and III procollagen mRNA, reduced by 49% and 91% respectively, compared to fibroblasts growing on plastic. See also, Eckes B. et al., “Downregulation of collagen synthesis in fibroblasts within three-dimensional collagen lattices involves transcriptional and posttranscriptional mechanisms,” FEBS Lett. 318:129-133 (1993) (observing that fibroblasts grown on collagen synthesize about 20% of the level of collagen they synthesize when grown on plastic).

By Northern blot analysis, oxandrolone enhances the expression of mRNA specific for α1(I) and α1(II) procollagen chains, in fibroblasts residing on a collagen matrix. Specifically, oxandrolone increases the expression of type III procollagen mRNA 11 fold and doubles the expression of type I procollagen mRNA in fibroblasts maintained on collagen, relative to control fibroblasts maintained on collagen. In contrast, fibroblasts growing on plastic and receiving oxandrolone (3 μg/ml) showed a minimal change in procollagen mRNA expression. A collagen substrate, such as a collagen matrix found in a wound, inhibits types I and III procollagen expression in fibroblasts. Compounds according to the present invention antagonize such collagen substrate inhibition of procollagen expression, stimulating procollagen expression to levels of fibroblasts growing on plastic.

The results in vitro are indicative of the effectiveness of compounds according to the present invention to enhance wound healing by increasing expression of procollagen in vivo. Given the guidance provided herein, those skilled in the art will understand that a wound or wounds may be treated by a method comprising the administration of a therapeutically effective amount of a compound which was identified by determining whether a compound of interest increases the expression of type I procollagen and/or type III procollagen by fibroblasts cultured in the presence of collagen.

Preferably, a compound that was identified by determining whether a compound of interest increases the expression of type I procollagen and/or type III procollagen by fibroblasts cultured in the presence of collagen is administered in an amount effective to increase healing of a wound or wounds in a patient. In certain circumstances, an identified compound is administered in an amount effective to increase healing of a wound or wounds in a patient without resulting in significant weight gain.

A wound or wounds may be treated by a method comprising the administration of a therapeutically effective amount of an anabolic steroid selected to increase expression of procollagen by fibroblasts. Preferably, the selected anabolic steroid is administered in an amount effective to increase healing of a wound or wounds in a patient. In certain circumstances, anabolic steroid is administered in an amount effective to increase healing of a wound or wounds in a patient without resulting in significant weight gain.

Therapeutically effective doses will vary, depending on such factors as the weight and condition of the patient, the severity and condition of the wound or wounds to be healed, and the method of administration. For example, higher doses may be required when a compound is administered orally as compared to when it is administered intravenously. Further, the amount of compound administered to achieve wound healing in a person not suffering from chronic involuntary weight loss, a catabolic state, or the like, may be a lesser amount than that required to achieve the same effect in a patient who is suffering from such a condition. The amount of compound administered per day to the local areas of a wound or wound will generally be lower than the dose that would be required to achieve the same effect if administered systemically. However, when an compound is administered in a systemic formulation in an effective amount, determination of the concentration of the compound in the local area of the wound or wounds to be treated may be useful in determining therapeutically effective doses for local administration.

Therapeutically effective doses of compounds may be determined by those skilled in the relevant arts via clinical studies by monitoring and comparing the rate of healing of wounds of patients treated with differing concentrations of the compound, including no compound (control). Other known indicators of wound healing may also be assessed to determine therapeutically effective doses of compounds according to the present invention. Such indicators include wound tensile strength, hydroxyproline or collagen content, procollagen expression, and re-epithelialization. Likewise, therapeutic concentrations of collagen in a wound are those concentrations which increase or improve wound healing according to accepted indicators.

Therapeutically effective doses may also be determined by studying the rate of healing of wounds of rats treated with differing concentrations of the compound, including no compound (control). Dosing information determined in rats may be extrapolated to humans using known techniques. For example, for systemic administration, the amount of compound administered per unit body weight determined in rats can easily be applied to humans. As another example, for local administration, the amount of compound administered per unit area of wound can be easily applied to wounds in humans.

Wound healing may be measured by many procedures known in the art, including wound tensile strength, hydroxyproline or collagen content, procollagen expression, and re-epithelialization.

As a specific example, oxandrolone may administered orally at a dosage of about 2.5-20 mg per day. As another specific example, oxandrolone may be administered orally at a dosage of about 0.1-0.2 mg/kg per day.

Therapeutic effectiveness may be measured as effectiveness in enhancing wound healing. Enhanced wound healing may be measured by known techniques such as decrease in healing time, increase in collagen density, increase in hydroxyproline, reduction in complications, increase in tensile strength, and increased cellularity of scar tissue.

Oxandrolone as used herein encompasses 17-methyl-17-hydroxy-2-oxa-5-androstan-3-one (both racemic mixtures and optically active enantiomers) as well as pharmaceutically acceptable esters thereof. For example, an oxandrolone product which is commercially available is the Oxandrin® tablet from BTG Pharmaceuticals Corp., Iselin, N.J. 08830. Oxandrin® comprises 17α-methyl-17β-hydroxy-2-oxa-5α-androstan-3-one. Methods of formulating compositions for administration are well know in the art, particularly the arts of pharmaceuticals and clinical medicine. See, e.g., Remington, The Science and Practice of Pharmacy, Alfonso R. Gennaro (Ed.) Lippincott, Williams & Wilkins (pub).

Compositions according to the present invention may be administered orally, intravenously, intramuscularly, subcutaneously, topically, intratracheally, intrathecally, intraperitoneally, rectally, vaginally, or intrapleurally.

If compositions according to the present invention are administered orally, they may be administered in the form of a tablet, a pill, a liquid or a capsule. Liquid formulations comprising compounds according to the present invention may also be formulated as sprays, which may be formulated to be suitable for, e.g., spraying into the mouth or spraying onto a wound and/or the surrounding area.

Compositions according to the present invention may be administered as buccal, lingual, or sublingual tablets, capsules, or lozenges.

A liquid may be administered in the form of a solution or a suspension. The liquid dosage form may comprise, for example, an alcohol-base or may be formulated with a cyclodextrin such as hydroxypropyl-β-cyclodextrin.

Compositions produced in accordance with the invention may comprise conventional pharmaceutically acceptable diluents or carriers. Tablets, pills, liquids and capsules may include conventional excipients such as lactose, starch, cellulose derivatives, hydroxypropyl methylcellulose and magnesium stearate. Conventional enteric coatings may also be used.

Dosage forms such as oral, rectal, and vaginal may be formulated for immediate release or for delayed release. Likewise, such dosage forms may be in a sustained-release formulation or in a once a day formulation.

Suppositories may include excipients such as waxes and glycerol. Injectable solutions will comprise sterile pyrogen-free media such as saline and may include buffering agents, stabilizing agents, solubilizing agents or preservatives.

Compositions for topical administration may be in the form of creams, ointments, lotions, solutions, transdermal delivery systems, transdermal patches, foams, or gels.

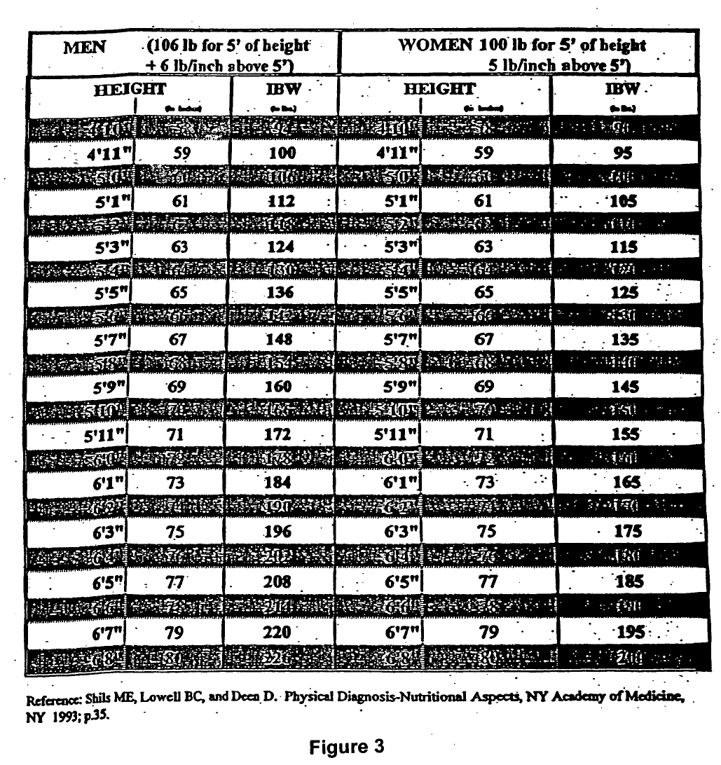

The ideal weigh of a patient may be determined by methods standard in the art of clinical medicine. As an example, an ideal weight chart, as in FIG. 3, may be used. As another example, a formula such as the following may be used. For a female, ideal weight is equal to 100 pounds for the first five feet of height, plus 5 pounds for each inch of height above five feet. For a male, ideal weight is equal to 106 pounds for the first five feet of height, plus 6 pounds for each inch of height above five feet.

### EXAMPLES

### Example 1

A primary cell line of normal human dermal fibroblasts (HF 250) was derived from the outgrowth of cells from explants of discarded foreskin. Fibroblasts were maintained in Dulbecco's modification of Eagles medium (DMEM) supplemented with 10% fetal bovine serum (FBS). Cells were grown to confluence and passed 1:1 by trypsinization. HF 250 cells were used between their 5^{th} and 7^{th} passages.

Acid-soluble collagen was isolated from rat tail tendons by acetic acid extraction. Briefly, rat tail tendons were teased from adult rat tails and stirred in ice cold 0.5 M acetic acid for 2 days. The solubilized collagen was cleared by centrifugation and the supernatant made 10% in respect to NaCl w/v. The precipitated collagen was collected by centrifugation, resuspended in 1 mM HCl, dialyzed exhaustively against 1 mM HCl, frozen, lyophilized, weighed, resuspended in sterile ice cold 1 mM HCl at 5 mg/ml and stored at 4° C. until needed. The bottoms of 10 cm Petri dishes were covered with cold, sterile collagen solution. Any free collagen solution was removed with a Pasteur pipette and the coated dishes placed in a 37° C. incubator, where they were incubated overnight to allow complete polymerization of the collagen-coated surface.

To optimize collagen synthesis and processing by cultured fibroblasts, the culture medium was supplemented with 50 μg/ml of vitamin C as L-ascorbic acid 2-phosphate sesquimagnesium salt (Sigma Chemical Co., St. Louis, Mo.). Oxandrolone was the anabolic steroid tested, and it was supplied by Bio-Technology General Corp., Iselin, N.J. A stock solution of oxandrolone was made in a 10% dimethyl-sulfoxide solution.

An initial pilot study was conducted to determine the concentration of anabolic steroid that would maximize collagen synthesis, as follows. After fibroblast cultures became confluent in 2 days, the culture medium was replaced with a medium supplemented with ascorbate at 50 μg/ml. To each dish, a different concentration of oxandrolone was added (0, 1, 3, 10 or 30 μg/ml). Dishes were incubated for 24 hours in a water saturated atmosphere at 37° in 95% air, 5% CO₂. To evaluate the level of collagen produced by fibroblasts in culture, collagen was extracted from the cell monolayer by limited proteinase digestion. All medium was removed, 2 ml of ice cold 0.5 M acetic acid with 0.2 mg of pepsin was added and the dishes incubated at 4° C. for 48 hours with agitation. To inactivate the pepsin, enough 1 M NaOH was added to neutralize the acetic acid extraction solution. Equal aliquots of pepsin-solubilized collagen were subjected to SDS polyacrylamide gel electrophoresis (SDS-PAGE). The intensities of Coomassie Brilliant Blue-stained protein bands in SDS-PAGE gels of denatured collagen α chains were compared to each other to determine the optimal level of anabolic steroid for stimulating collagen synthesis. The maximal intensity of collagen a chain staining was obtained when 3 μg/ml of oxandrolone was added to cultures (data not shown). All subsequent treated cell cultures received 3 μg/ml of oxandrolone.

### Example 2

Equal numbers of fibroblasts were plated onto tissue culture plastic dishes or collagen-coated Petri dishes. Four confluent 10 cm dishes of HF 250 cells were split in half and the cells replated in four 10 cm collagen-coated and four uncoated dishes. Two collagen-coated dishes and two uncoated dishes received 3 μg/ml of oxandrolone. The two remaining collagen-coated and uncoated dishes received an equal volume of 10% dimethyl-sulfoxide alone. After 24 hours of incubation, the medium was removed and the dishes rinsed with phosphate buffered saline. Northern blot analysis was used to determine the levels of both type I and type III procollagen mRNA expression. Total RNA was extracted from culture cells, using the Total RNA Isolation kit (Ambion, Inc; Austin, Tex.) following the supplier's instructions. Type I and type III procollagen cDNAs were purchased from American Type Culture Collection (Manassas, Va.). The human α₁ (I) (HF677) and α₁ (III) (HF934) procollagen cDNAs were enzymatically cleaved, purified, and subsequently end-labeled with ³²P dCTP (New England Nuclear, Boston, Mass.) to generate specific cDNA probes.

Total extracted RNA was subjected to agarose gel electrophoresis and blotted on a nylon membrane. After incubating the membrane with the probes, the membrane was washed, processed and exposed to x-ray film. The x-ray film was developed and quantitative analysis done by densitometry. To confirm uniform RNA loading of each sample on the membrane, a gluteraldehyde-3-phosphate dehydrogenase (GAPDH) probe was employed to reprobe the membrane. The quantities of type I and type III procollagen α₁ mRNAs and GAPDH mRNA were determined by densitometry. The ratio of these densities were compared and reported as ratios of procollagen mRNA/GAPDH mRNA densities.

By 24 hours, the density of cells on the plastic surface appeared greater than the density of cells grown on collagen. Human dermal fibroblasts plated on collagen have previously been shown to exhibit retarded cell division. See, e.g., Greco and Ehrlich (1992). The inclusion of oxandrolone did not affect the number of fibroblasts grown on collagen or on plastic. There was no obvious difference in cell morphology.

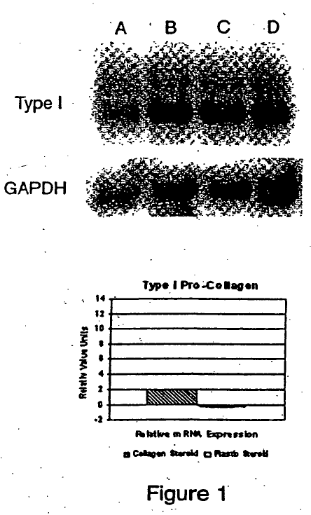

As shown in FIG. 1, the endogenous expression of type I procollagen mRNA expressed in fibroblasts grown on collagen (lane A) was less than the endogenous expression of mRNA expressed in fibroblasts grown on plastic (lane C). The densities of the GAPDH bands show variations in RNA loading (middle panel). Oxandrolone doubled the level of mRNA for type I procollagen in cells grown on collagen as compared to cells receiving 10% dimethyl sulfoxide alone (lane B). Oxandrolone did not noticeably change the expression of type I procollagen mRNA in cells growing on plastic as compared to controls (compare lane C to D).

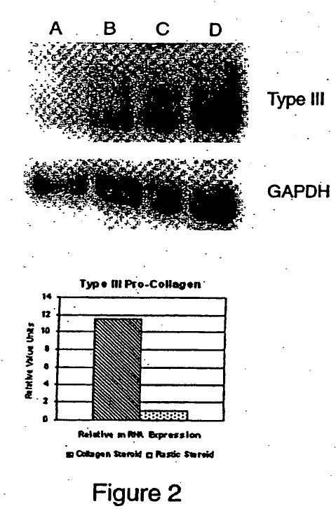

As shown in FIG. 2, the endogenous expression of type III procollagen mRNA in fibroblasts grown on collagen was greatly reduced compared to fibroblasts maintained on plastic (lanes A and C). When oxandrolone was included, the expression of type III procollagen mRNA dramatically increased 11 fold for fibroblasts grown on collagen (lane B) relative to cells grown on collagen receiving 10% dimethyl sulfoxide (lower panel). When fibroblasts were grown on plastic, the addition of oxandrolone (lane D) had a minimal effect upon type III procollagen mRNA expression in comparison to control cells grown on plastic receiving 10% dimethyl sulfoxide (lane C). Oxandrolone increased the expression of mRNA for both type I and type III procollagens in fibroblasts maintained on collagen surfaces, but had a minimal effect on fibroblasts grown on plastic. It appears that fibroblasts on plastic are expressing near maximal levels of procollagen mRNA.

1. A method for identifying a compound useful for the treatment of wounds comprising determining whether the compound increases the expression of type I procollagen and/or type III procollagen by human fibroblasts cultured in the presence of collagen.

2-73. (canceled)

74. The method of claim 1, further comprising determining whether the compound does not substantially increase the expression of type I procollagen and/or type III procollagen by human fibroblasts cultured in the presence of plastic.

75. The method of claim 1 or 74, wherein the compound increases the expression of type I procollagen about two-fold.

76. The method of claim 1 or 74, wherein the compound increases the expression of type III procollagen about eleven-fold.

77. The method of claim 1 or 74, wherein the compound increases the expression of type I procollagen about two-fold and increases the expression of type III procollagen about eleven-fold.
